(19) United States
(12) Patent Application Publication
(10) Pub. No.: US 2008/0319417 A1
(43) Pub. Date: Dec. 25, 2008

(54) Cells isolation system for breast augmentation and reconstruction

(76) Inventors: Rodolfo C. Quijano, Laguna Hills, CA (US); Hosheng Tu, Newport Beach, CA (US)

Correspondence Address: HOSHENG TU, 15 RIEZ, NEWPORT BEACH, CA, 92657-0116, US

(21) Appl. No.: 11/821,155

(22) Filed: Jun. 22, 2007

(51) Int. Cl. A61M 31/00; A61M 1/00

(52) U.S. Cl. 604/522; 604/542

(57) ABSTRACT

A one-step real-time system for treating, reconstructing or augmenting a breast tissue defect in a patient comprising means for extracting adipose tissue, isolate adipocytes, process and re-inject stem and regenerative cells to the breast of the patient.

### FIELD OF THE INVENTION

The present invention is related to a breast augmentation system for treatment of breast tissue defect, more particularly, the present invention relates to a real-time adipose cells isolation system employed for injecting purified adipose stem and regenerative cells to repair, augment or reconstruct a breast tissue defect in a patient.

### BACKGROUND OF THE INVENTION

Adipose tissue, also known as fat tissue, contains a specialized class of stem cells, which are comprised of multiple cell types that might promote healing and repair. It appears that adipose-derived stem cells home in on specific sites of injury through biological signaling that occurs naturally.

In anatomy, adipose tissue or fat is loose connective tissue composed of adipocytes. Its main role is to store energy in the form of fat, although it also cushions and insulates the body. Obesity in animals, including humans, is not dependent on the amount of body weight, but on the amount of body fat—specifically adipose tissue. In mammals, two types of adipose tissue exist: white adipose tissue (WAT) and brown adipose tissue (BAT). Adipose tissue also serves as an important endocrine organ by producing hormones such as leptin, resistin and the cytokine TNFα.

Adipose tissue is primarily located beneath the skin, but is also found around internal organs. In the integumentary system, which includes the skin, it accumulates in the deepest level, the subcutaneous layer, providing insulation from heat and cold. Around organs, it provides protective padding. It also functions as a reserve of nutrients. In a severely obese person, excess adipose tissue hanging downward from the abdomen is referred to as a panniculus (or pannus). A panniculus complicates surgery of the morbidly obese. The panniculus may remain as a literal “apron of skin” if a severely obese person quickly loses large amounts of weight (a common result of gastric bypass surgery). The condition can be corrected with proper diet and exercise. It is a misconception that plastic surgery is the only way to fix the problem. Adipose tissue has an “intracellular matrix,” rather than an extracellular one. Adipose tissue is divided into lobes by small blood vessels. The cells of this layer are adipocytes.

Free fatty acid is “liberated” from lipoproteins by lipoprotein lipase (LPL) and enters the adipocyte, where it is reassembled into triglycerides by esterising it onto glycerol. Human fat tissue contains about 87% lipids. Fat cells have an important physiological role in maintaining triglyceride and free fatty acid levels, as well as determining insulin resistance. Abdominal fat has a different metabolic profile—being more prone to induce insulin resistance. This explains to a large degree why central obesity is a marker of impaired glucose tolerance and is an independent risk factor for cardiovascular disease even in the absence of diabetes mellitus and hypertension.

Recent advances in biotechnology have allowed for the harvesting of adult stem cells from adipose tissue, allowing stimulation of tissue regrowth using a patient's own cells. The use of a patient's own cells reduces the chance of tissue rejection and avoids the ethical issues associated with the use of human embryonic stem cells. In general, a stem cell shows ability of a clonal stem cell population to self-renew, ability of a clonal stem cell population to generate a new, terminally differentiated cell type in vitro and ability of a clonal stem cell population to replace an absent terminally differentiated cell population when transplanted into an animal depleted of its own natural cells.

Important parts of the breasts include mammary glands, the axillary tail, the lobules, Cooper's ligaments, the areola and the nipple. As breasts are mostly composed of adipose tissue, their size can change over time if the woman gains or loses weight. For those women with breast defect, it is desirable to transplant stem cells or certain biomatrix containing stem cells via re-infusing to repair or augment the breast tissue defect.

Whereas embryonic stem cells are the building blocks for all of the cell types in the body, adult stem cells are a more specialized type of progenitor cell. Adult stem cells are found in specific tissues and have the ability to regenerate themselves, as well as differentiate into all of the cell types found in that tissue. The specific differentiation pathway that these cells enter depends upon various influences from mechanical influences and/or endogenous bioactive factors, such as growth factors, cytokines, and/or local microenvironmental conditions established by host tissues. Using cells from the developed individual, rather than an embryo, as a source of autologous stem cells would overcome the problem of tissue incompatibility associated with the use of transplanted embryonic stem cells.

Fat injections in the breasts have always been taboo, because large globules of fat can calcify and resemble cancerous tumors on mammograms. Recently, it was reported that microdroplets of autologous fat was injected to the breast using tiny needles after priming breasts with a suction device to increase blood supply. Six months later, MRIs found 90% of the fat still present with only minimal calcification, which was detectable as noncancerous. It is presently accepted that early and adequate revascularization of injected adipose slurries could be a factor in breast tissue regeneration and survival processes.

Adipose tissue offers a potential source of multipotential stromal stem cells. Adipose tissue is readily accessible and abundant in many individuals. Obesity is a condition of epidemic proportions in the United States, where over 50% of adults exceed the recommended BMI based on their height. Adipocytes can be harvested by liposuction on an outpatient basis. This is a relatively non-invasive procedure with cosmetic effects that are acceptable to the vast majority of patients. It is well documented that adipocytes are a replenishable cell population. Even after surgical removal by liposuction or other procedures, it is common to see a recurrence of adipocytes in an individual over time. This suggests that adipose tissue contains stromal stem cells that are capable of self-renewal.

There is an unmet clinical need to develop an effective one-step autologous system that extracts fat from a patient, isolates the adipose stem cells and regenerative cells, processes and purifies the cells, and injects the purified cells into the breast defect of the same patient.

### SUMMARY OF THE INVENTION

One object of the invention is to provide a real-time one-step system and method for aspiring (extracting), isolating, purifying, and concentrating adipose stem and regenerative cells from a donor patient and injecting derived adipose stem and regenerative cells optionally with cell carrier to repair, augment or reconstruct a breast tissue defect to the donor patient in an autologous manner. In one embodiment, one object of the system is for breast enhancement or enlargement.

Some aspects of the invention provide a system of treating a breast of a patient, the system comprising: a) means for fat tissue extraction from a body of the patient, wherein the extracted fat tissue comprises adipocytes; b) means for isolating the adipocytes from the extracted fat tissue; c) means for compounding the adipocytes with a cell carrier; and d) means for re-injecting the compounded adipocytes to the breast of the patient.

The breast in the system of treating a breast of a patient of the present invention is a breast with defect, wherein the defect may be created mechanically, chemically, or electromagnetically.

The system of treating a breast of a patient comprises means for compounding the adipocytes with a cell carrier, wherein the cell carrier is a free-flowing gel-like biodegradable support element. In one preferred embodiment, the cell carrier and the adipocytes is compounded and configured to transiently, form a confluent cell-sheet like configuration. In another embodiment, the confluent cell-sheet like configuration is being formed after the compound being injected into the breast.

The system of treating a breast of a patient comprises means for compounding the adipocytes with a cell carrier, wherein the cell carrier is collagenous extracellular matrix, wherein the cell carrier further comprises at least one growth factor. In one embodiment, the system of treating a breast of a patient comprises means for compounding the adipocytes with a cell carrier, wherein the cell carrier is a temperature-sensitive deformable gel material, such as methylcellulose. In one embodiment, the system of treating a breast of a patient comprises means for compounding the adipocytes with a cell carrier, wherein the cell carrier is a pH-sensitive deformable gel material, such as chitosan/alginate complex.

The system of treating a breast of a patient comprises means for fat tissue extraction from a body of the patient, wherein the extracted fat tissue comprises adipocytes, and wherein means for fat tissue extraction from the body of the patient is a liposuction process or suction assisted lipectomy.

The system of treating a breast of a patient comprises means for isolating the adipocytes from the extracted fat tissue using a centrifugal separation process or a non-centrifugal separation process, wherein the non-centrifugal separation process is operated with an orbital motion mode.

The system of treating a breast of a patient comprises means for isolating the adipocytes from the extracted fat tissue in a separation process with an alternate centrifugal separation mode and a non-centrifugal separation mode.

Some aspects of the invention provide a real-time process of treating a breast of a patient, the process using a system that comprises: a) means for fat tissue extraction from a body of the patient, wherein the extracted fat tissue comprises adipocytes; b) means for isolating the adipocytes from the extracted fat tissue; c) means for compounding the adipocytes with a cell carrier; and d) means for re-injecting the compounded adipocytes to the breast of the patient in an autologous manner.

### BRIEF DESCRIPTION OF THE DRAWINGS

Additional objects and features of the present invention will become more apparent and the disclosure itself will be best understood from the following Detailed Description of the Exemplary Embodiments, when read with reference to the accompanying drawings.

FIG. 1 shows a schematic diagram of a system for treating the breast defect of a tissue donor.

FIG. 2 shows a schematic diagram of a hybrid cells separation process employing a separation chamber under an orbital motion and/or a rotational motion of the present invention.

FIG. 3 shows an illustrative setup of a hybrid cells separation process employing a separation chamber comprising a filter membrane under an orbital motion and/or a rotational motion.

FIG. 4 shows a bottom view of the cells separation apparatus comprising the separation chamber having a filter membrane.

FIG. 5 shows a perspective view of the cells separation apparatus comprising the separation chamber having a filter membrane.

FIG. 6 shows one embodiment of a cell isolation system of the present invention, including a stand-alone enclosure, an inlet port for receiving aspired fat tissue, an outlet port for providing injectable cell compound and external power sources.

FIG. 7 shows a front view of a centrifuge sub-assembly unit.

FIG. 8 shows a top view of the centrifuge sub-assembly unit in FIG. 7.

FIG. 9 shows a complete set of a centrifuge assembly unit.

FIG. 10 shows a simulated process of transferring a centrifuge sub-assembly unit from the cell centrifuge set to the cell compounding set.

FIG. 11 shows a complete set of a cell compounding assembly unit.

FIG. 12 shows an illustrative cell compounding sub-assembly unit.

FIG. 13 shows various embodiments for a cell injector or administrator to the patient.

FIG. 14 shows a preferred embodiment for a cell injector to the patient.

### DETAILED DESCRIPTION OF THE EXEMPLARY EMBODIMENTS

The preferred embodiments of the present invention described below relate to a system, particularly to a real-time system without culturing or storage delay, for procuring fat tissue from a patient who is a tissue donor and simultaneously a recipient, isolating adipocytes and regenerative cells, processing the cells via biomarker identification, purification and compounding, and re-injecting the desired stem and regenerative cells compound back to the patient by optionally adding biomatrix substrate or cell carrier(s). The cells produced by the processes of the invention are useful in providing a source of fully functional cells for tissue regeneration to treat human breast defect, to repair and to augment a breast. In one aspect, the invention provides a biomatrix (or cell carrier) for differentiating adipocytes or adipose tissue-derived stromal cells into breast tissue cells in vivo that comprises a medium capable of supporting the growth and differentiation of stromal cells into functional breast cells after injection. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

Stem cells are becoming more widely used in plastic surgery for breast augmentations, breast enlargement and to enlarge any body part where more fullness is sought. With the European Union and Britain having just approved the use of stem cells for cosmetic surgery recently, this move is likely to make the use of stem cells more and more popular worldwide and ultimately in the United States.

Stem cells have been used in breast augmentations since 2003, as Japanese scientists have pioneered a treatment that offers a natural breast augmentation that uses stem cells and fat derived from the patient's own body to create soft and naturally augmented breasts. As no implants are needed and only self-extracted stem cells and fat are used, the patient's enhanced breasts are essentially “real.” This procedure has been performed on several patients (for breast enhancement and facial rejuvenation procedures) thus far without any patients reporting any major problems.

In a prior clinical report, one stem cell-assisted treatment could successfully increase breast volume by 120-160 ml, which is the rough equivalent of two bra-cup sizes (5 to 7 cm). For patients seeking to augment their breasts by 300 ml, the treatment needs to be performed twice. In both cases, no implant is used. For augmentations exceeding 300 ml, a combination of an implant and the stem cell technique is used to achieve the desired results.

The technique is performed by suctioning fat from the abdomen, omentum or thigh and then injecting the fat together with adipose-derived stem cells obtained from the patient back into the breast. The liposuctioned fat mixture, which now contains a high level of stem cells, is then transplanted layer by layer back into each breast to ensure an even distribution of the fatty mixture. What then occurs is that the stem cells enable the fat to grow its own blood supply, which leads to the fat becoming a part of the breast as opposed to a foreign mass. Some of the cells produce more fat and other cells change into a living blood supply for new breast tissue that grows into the treated breast. Unlike traditional breast augmentations, there are no incisions involved, as only small needle punctures are made on each breast that lead to tiny imperceptible marks.

By “progenitor” or “regenerative cell” it is meant an oligopotent or multipotent or often referred to as “pluripotent” stem cell which is able to divide without limit and, under specific conditions, can produce daughter cells which terminally differentiate such as into breast cells. These cells can be used for transplantation into a heterologous, autologous, or non-autologous host. By heterologous is meant a host other than the animal from which the progenitor cells were originally derived. By autologous is meant the identical host from which the cells were originally derived. During the cell processing, cell suspensions in medium may be supplemented with certain specific growth factor that allows for the proliferation of target progenitor cells after their delivery to the recipient. The medium for cells suspension is also considered one type of cell carrier or biometric.

By “adipose” is meant any fat tissue. The adipose tissue may be brown or white adipose tissue, derived from subcutaneous, omental/visceral, mammary, gonadal, or other adipose tissue site. A convenient source of adipose tissue is from liposuction surgery, however, the source of adipose tissue or the method of isolation of adipose tissue is not critical to the invention. When stromal cells are desired for autologous transplantation into a subject, the adipose tissue will be isolated from that subject and administered to the specific breast defect site for tissue regeneration, for example, in a one-step and/or real-time system.

Liposuction is the most frequently performed procedure in plastic surgery. Liposuction and/or suction-assisted lipectomy remove fat cells from parts of the body where excess fat cells exist. The liposuction procedure involves making one or more small poke wounds in areas like the abdomen, hips or thighs. Through these small incisions, a long metal tube (a cannula), with small hole(s) at one end section and connected to one atmosphere of negative pressure at the other end, is inserted. The cannula, in the 3-5 mm diameter range, is repeatedly moved in and out of the surgical site. A network of holes, like a sponge or Swiss cheese, is made in the bulging area and the fat is liquefied and removed. Sometimes, ultrasound vibrational energy is added to enhance the fat emulsification (ultrasound-assisted liposuction). Afterwards, the overlying skin is compressed with a binder or girdle to tighten the tissues for a couple of weeks. During the procedure, the area to be suctioned is filled with isotonic saline solution, local anesthetic, and vasoconstrictor. The saline serves to emulsify and soften the fat and makes it easier to remove.

The vacuum pump for small cannula should develop vaporization pressure, and it must have a receptacle large enough to collect as much as 2 L of fatty tissue before entering the reservoir. For example, a ⅓ hp motor coupled with a 60 L/min, two-stage, oil-type rotary pump could produce a vapor pressure vacuum of 743 mmHg in a 2.5-L collection jar in 15 seconds. The pumps used in suction-assisted lipectomy and involved in the present real-time cell isolation system for breast treatment may include piston-type vacuum pump, diaphragm pump, rotary pump, or the like. However, the simplest vacuum system consists of a syringe with a cannula attached where the needle would normally be. When the air space in the cannula is displaced with a small amount of sterile solution and the plunger is withdrawn, the space created is close to maximum vacuum. A mechanical lock to hold the plunger open is necessary.

Variable vacuum is a means of controlling the desired vacuum. It is generally done by controlling a small orifice to blend air into the system and can be programmed via a computer CPU for optimal auto-lipectomy in the real-time cell isolation system.

Any medium capable of supporting stromal cells in tissue suspension (or tissue slurry) may be used as cell carrier of the present invention, for example, Dulbecco's Modified Eagle's Medium that supports the growth of fibroblasts. Growth factors are generally added to the medium for supporting stromal cells in tissue suspension. Typically, 0 to 20% Fetal Bovine Serum (FBS) is added to the above medium in order to support the growth of stromal cells.

Non-limiting examples of media useful in the methods of the invention can contain fetal serum of bovine or other species at a concentration of at least 1% to about 30%, preferably at least about 5% to 15%, mostly preferably about 10%. Embryonic extract of chicken or other species can be present at a concentration of about 1% to 30%, preferably at least about 5% to 15%, most preferably about 10%.

The growth factors of the invention may include, but not limited to, transforming growth factor-β (TGF-β1, TGF-β2, TGF-β3 and the like), insulin-like growth factor, platelet derived growth factor, epidermal growth factor, acidic fibroblast growth factor, basic fibroblast growth factor, hepatocytic growth factor, and the like. The concentration of growth factors is about 1 to about 100 ng/ml. In one embodiment, the biomatrix for incorporating the stromal cells is a component of the collagenous extracellular matrix such as collagen I (particularly in the form of a gel). Other nutrient, such as vitamin A, vitamin A analogue (such as retinoic acid), vitamin B series, vitamin C, and vitamin C analogue or other vitamins may be added to the medium. The concentration of retinoic acid or other nutrient is about 0.1 to 10 μg/ml.

The present invention also provides a method for formulating adipose derived stromal cells, in absence of an in vitro culture step, with a biocompatible pharmaceutical carrier for injecting into the breast of a subject. In one embodiment, the biocompatible carrier, or biomatrix (that is, cell carrier) as defined herein, may be in the form of slurry, gel, a malleable gel, colloid, solution, or suspension. A process for manufacturing an injectable cell-seeded gel material may comprise the steps of: providing a biocompatible carrier and stem cells source; combining the cells and the carrier in a uniformly dispersed form; and applying a pressurizing force to the combined gel material for injecting into the breast of the subject.

Tissues are highly organized in their geometry and architecture with respect to how cells are positioned relative to each other, as well as to the surrounding soluble factors and extracellular matrix molecules within a given microenvironment. U.S. patent application publication no. 2007/0092493 A1, entire contents of which are incorporated herein by reference, discloses a method of preparing a 3-D living cell construct comprising coating a thermoreversible hydrogel on a 3-D scaffold support element, wherein the hydrogel comprises methylcellulose, phosphate buffered saline, and collagen; loading target living cells onto the support element (a process called ‘compounding’); incubating the support element for a predetermined duration (an optional step that might be skipped), wherein the support element is biodegradable. In one aspect of the invention, the stem cells seeded gel material of the present invention comprises target living stem and regenerative cells loaded with a gel-like biodegradable support element, wherein the stem/regenerative cells continue to transiently form confluent cell-sheet like configuration with enhanced cell proliferation in vivo under the presence of the support element after the compound being injected into the breast. In one embodiment, the gel-like biodegradable support element of the present invention may comprise collagenous extracellular matrix, preferably with growth factor(s) or nutrients. In another embodiment, the gel-like biodegradable support element may comprise chitosan, collagen, elastin, gelatin, fibrin glue, biological sealant, polymers, or combinations thereof.

The adipose tissue derived stem cells or regenerative cells useful in the methods of invention may be isolated by a variety of methods known to those skilled in the art. In a preferred method, adipose tissue is isolated from a human subject. A preferred source of adipose tissue is omental adipose. In humans, the adipose is typically isolated by liposuction. If the cells of the invention are to be transplanted into a human subject, it is preferable that the adipose tissue be isolated from that same subject so to provide for an autologous transplant. Alternatively, the administered tissue may be allogenic.

The present invention provides a method of repairing damaged tissue in a human subject in need of such repair by delivering the isolated multipotent adult stem cells to the damaged tissue of the subject. The cells may be introduced into the body of the subject by localized injection. The cells may be introduced into the body of the subject in conjunction with a suitable biomatrix. The cells may be introduced into the body of the subject with a portion of the cells in a confluent configuration. The biomatrix may incorporate and provide additional genetic material, cytokines, growth factors, or other factors to promote growth and differentiation of the cells in vivo after being injected to the breast of a recipient, autologously or allogenically.

In one embodiment of the invention, an adipose tissue derived stem or regenerative cell is induced to express at least one phenotypic characteristic of breast tissue cell. Phenotypic markers of the desired cells are well known to one ordinary skilled in the art, and copiously published in the literature. Additional phenotypic markers continue to be disclosed or can be identified without undue experimentation. Any of these markers can be used to confirm that the adipose cell has been induced to a differentiated state after being injected in a related animal model, if needed. Lineage specific phenotypic characteristics can include cell surface proteins, cytoskeletal proteins, cell morphology, and secretory products. Some aspects of the invention provide adipose tissue-derived stromal cells that exhibit the improved properties of increased extracellular matrix proteins and/or a lower amount of lipid than a mature isolated adipocyte.

In another embodiment, the biocompatible cell carrier (preferably configured for cells to home in) or biomatrix may be a temperature-sensitive or pH-sensitive deformable gel material that forms shaped construct, structure, or 3-dimensional scaffold upon delivered to the target breast site in vivo and the scaffold reaches the threshold temperature or pH. Examples of biocompatible carrier material include alginate, agarose, fibrin, collagen, chitosan, gelatin, elastin, and combinations thereof. In one embodiment, the biocompatible cell carrier is preferably biodegradable or bioresorbable. Examples of biodegradable matrix material may include, but not limited to, polymers or copolymers of lactide, glycolide, caprolactone, polydioxanone, and trimethylene carbonate. Examples of biodegradable matrix material may also include polyorthoesters and polyethylene oxide. The pH-sensitive hydrogel biomatrix may include N-akylated chitosan, chitosan/alginate complexes, and the like. The temperature-sensitive hydrogel biomatrix may include methylcellulose. Literature shows that methylcellulose solutions could reveal a low viscosity at room temperature (suitable for free flowing) and formed a soft gel at 37° C.; thus making methylcellulose well suited as an injectable carrier for stem and regenerative cells for the repair of defects in the breast or breast augmentation.

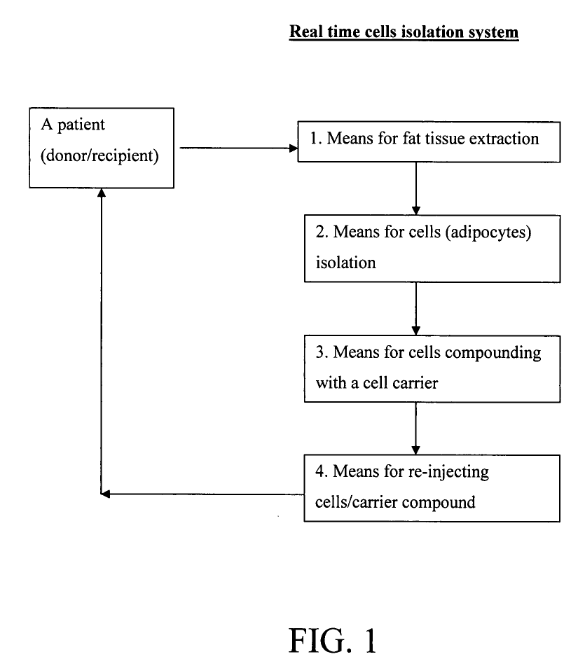

FIG. 1 shows a schematic diagram of a one-step and/or real-time system for treating a breast defect or breast enhancement. The system comprises: a) means for fat tissue extraction from a body of the patient, wherein the extracted fat tissue comprises adipocytes; b) means for isolating the adipocytes from the extracted fat tissue; c) means for compounding the adipocytes with a cell carrier; and d) means for re-injecting the compounded adipocytes to the breast of the patient.

An important issue concerning the therapeutic use of stem cells is the quantity of cells and cells colony necessary to achieve an optimal effect. In current human studies of autologous mononuclear bone marrow cells, empirical doses of 10 to 40×10⁶ are being used with encouraging results. In a study designed to treat peripheral vascular disease with autologous bone marrow, much larger doses were administered to the gastrocnemius muscle (2.7×10⁹ cells), with minimal inflammation and positive results (Lancet. 2002; 360:427-435). U.S. patent application publication no. 2007/0092493 A1, entire contents of which are incorporated herein by reference, discloses living cell sheets as an injectable cell substrate. In one embodiment, the stem cells or regenerative cells of the present invention configured in a substantial cell sheet format (or cell cluster with substantial cell-confluent configuration) is re-injected to the donor patient to treat breast defect or to augment the breast.

### EXAMPLE NO. 1 ONE-STEP SYSTEM

Some aspects of the invention relate to a system of treating a breast of a patient, the system comprising: a) means for fat tissue extraction from a body of the patient, wherein the extracted fat tissue comprises adipocytes and/or regenerative cells; b) means for isolating the adipocytes and regenerative cells from the extracted fat tissue; c) means for compounding the adipocytes and regenerative cells with cell carrier(s); and d) means for re-injecting the compounded adipocytes and regenerative cells to the breast of the patient. One aspect of the invention relates to a process for treating a breast defect using the system of the present invention in a real time manner and in an autologous manner. Another aspect of the invention relates to a method of treating a breast defect or breast augmentation in a patient, the method comprising: a) fat tissue extraction step; b) adipocytes and regenerative cells isolation step; c) cells compounding or processing step; and d) cells re-injecting step via administering the adipocytes and/or regeneration cells to a breast area in the patient. In one embodiment, the breast tissue defect is created (mechanically, chemically or electromagnetically) as an adjunct step before the cells re-injecting step for promoting stem cells differentiation and tissue regeneration at about the defect site after cells delivery in vivo. The method may be a one-step real-time (and/or automatic) system that can be performed in an operating room when the patient is on the operation table as both a donor of adipose tissue and a recipient of autologous stem and regenerative cells. An exemplary example of mechanically creating a defect in the breast is to insert a needle with a curved tip and move the needle tip around inside the breast. An exemplary example of chemically creating a defect in the breast is to inject a mild acid with a pH around 3-5 into the breast. An exemplary example of electromagnetically creating a defect in the breast is to expose the breast with some light source in the ultrasonic wavelength, radiofrequency wavelength or electromagnetic wavelength (either externally to a breast or in situ within a breast).

As illustrated in FIG. 1, the fat tissue extraction process or step may be carried out, for example by liposuction from a patient who may be a donor and a recipient in the above-referred autologous process. The adipocytes isolation process or step may include breakup of the fat mass and removal of the unwanted non-cellular material. The breakup of the fat mass may be accomplished by stirring or agitating in a mixer or juicer-like apparatus with added saline or other isotonic fluid. After breaking-up, the fat would suspend in the top layer while the cell-containing fluid is at the bottom portion of the apparatus. The cells isolation step may further comprise separating or filtering the desired stem and regenerative cells from other constituents. In another exemplary embodiment, the filtered stem and regenerative cells may be loaded with certain amount of fat and optionally with other biomatrix (nutrients, growth factors and other substance may be added to enhance cell differentiation into breast tissue cells) as a slurry-like or gel-like injectable substrate.

The cells isolation step may be accomplished by subjecting the fat-suspended adipose tissue solution to a centrifugal separation or other separation means. A non-centrifugal separation system was disclosed in U.S. Pat. Nos. 6,423,023, 6,690,178 and 6,969,367, that teaches a filtration system comprising: fluid supply means for supplying a fluid containing filtrate and particulate constituent; a filtration chamber having a hollow interior enclosed by a first plate, a second plate, and a flexible seal element between the fist plate and the second plate, wherein the first plate is essentially parallel to or at an acute angle to the second plate so as to form a chamber gap for the hollow interior; means for directing the fluid supply into the hollow interior; a non-rotational drive structure; the second plate comprising filter membrane means for separating filtrate from the particulate constituent, wherein the second plate is detachably coupled to the non-rotational drive structure that controls the second plate in an orbital motion in reference to a center axis of the first plate; a collecting means; filtrate collecting means for directing the filtrate passing through the filter membrane means to the collecting means; and particulate constituent collecting means for directing from the chamber gap a remaining constituent of the fluid supply out of the chamber.

The high-speed centrifugal separation system is generally too harsh to cell integrity while the above-cited non-centrifugal separation system is too mild to effect cells isolation and separation of the current fat-suspended adipose tissue solution. Some aspects of the invention relate to a hybrid separation system that combines the advantages of the two separation means (one with orbital motion and the other with rotational motion), resulting in unexpected separation efficiency for one-step adipose cells isolation system employed for injecting purified adipose stem and regenerative cells to repair, augment or reconstruct a breast tissue defect in a patient.

By “orbital motion” is meant herein as a motion that moves back and forth between two points in a continuous manner, wherein the route of the forward movement may either partially overlap or not overlap the route of the backward movement. However, the “orbital motion” is different from “rotation” in this patent application. By “rotation” is meant as a movement in such a way that all particles follow circles with a common angular velocity about a common axis.

### EXAMPLE NO. 2 HYBRID CELLS SEPARATION SYSTEM

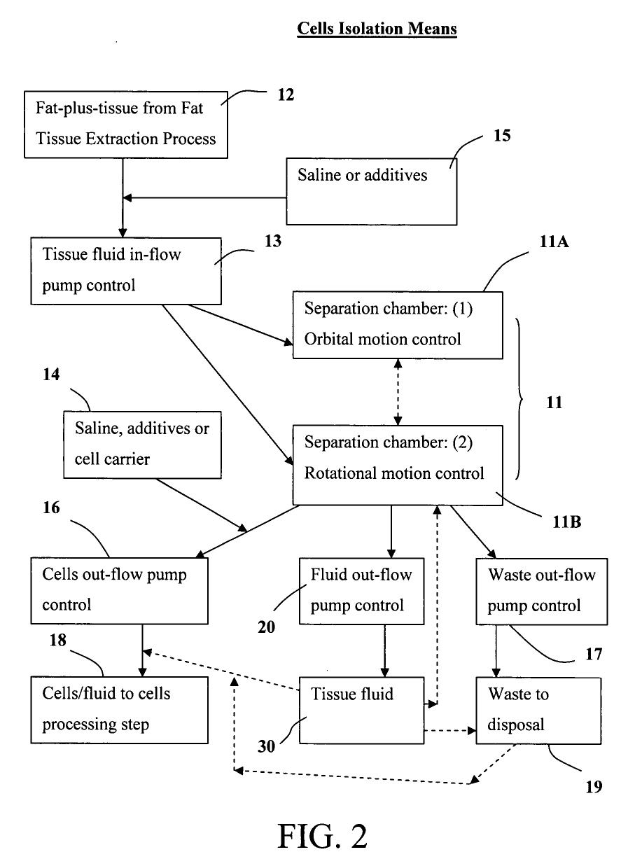

FIG. 2 is a schematic diagram of a hybrid cells separation process employing a hybrid separation chamber under (a) an orbital motion, (b) a rotational motion, and (c) combination of orbital motion and rotational motion of the present invention. The fat-plus tissue (12) via the fat extraction step is added with saline or additives (15) before being transported to the separation chamber (11). The fat-plus tissue may comprise fat, cells, blood vessels, liquid, and other solids. The fat-plus tissue is fed to the separation chamber (11) via a tissue fluid in-flow pump control (13) or other means for directing the fat/tissue material into the separation chamber (11). The separation chamber of the present invention may comprise a chamber with orbital motion control (11A), a chamber with rotational motion control (11B), or a chamber with orbital motion control followed by rotational motion control, or vice versa. Similarly, saline, additives, or cell carrier (14) may optionally be added after the cell isolation step. The additives may include nutrients, growth factors, biodegradable biomatrix, cell carrier(s), and the like. A positive pressure is generally maintained during the cell isolation process of the present invention. The pressure difference across the membrane is preferably in the range of 1 to 1000 mm of mercury, preferably 10 to 100 mm of mercury. The pressure difference is controlled by the flow rates of the tissue fluid in-flow pump control (13), the cells out-flow pump control (16), the fluid out-flow pump control (20), and optionally the fat-containing waste flow pump control (17).

Tissue fluid (30) is collected from the opposite side of the filter membrane, wherein the filtrate collecting means is completely isolated from communication with the tissue fluid in-flow supply. The filtrate is collected from the separation chamber (11) via a filtrate outflow pump control (20) or other means for directing the fluid constituent passing through the filter membrane. The cells component is retained on the adjacent surface of the separation membrane and exits the separation chamber via the cells out-flow pump control (16). Fat component or the residual constituent portion that floats at the top of the separation chamber is withdrawn from the separation chamber (11) via an optional waste flow pump control (17) or other means for directing a remaining constituent of the waste (19) out of the chamber. The pressure drops across the filter membrane can be adjusted by manually adjusting one or more of the flow pump controls (13, 16, 17, and 20), or by providing automatic adjusting mechanisms. The pressure drop may be measured by a differential pressure indicator and/or controlled by the automated adjusting mechanisms. Certain amount of tissue fluid (30) and fat waste (19) are added to the isolated cells (18) and forward the cells/fluid to cells processing step (18).

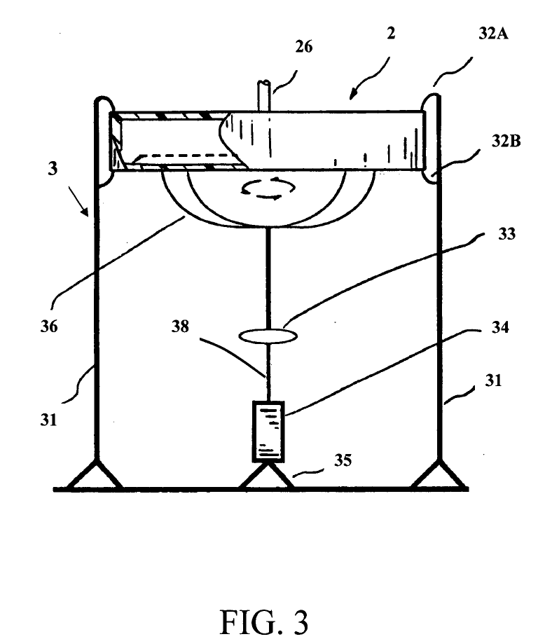

FIG. 3 shows an illustrative setup of a hybrid cells separation process employing a separation chamber comprising a filter membrane under an orbital motion and/or a rotational motion. A cells isolation setup comprises a supporting installation (3) that can be rolled away or placed at any convenient location and a removable cells separation apparatus (2). The supporting installation (3) comprises more than two supporting poles (31), preferably more than four, and a rotatable means for providing rotator action (34). The rotatable means would generate a rotational motion to the separation apparatus (2) when the shaft (38) is disengaged from the non-rotational element (33), whereas the rotatable means would generate an orbital motion to the separation apparatus (2) when the shaft (38) is engaged to the non-rotational element (33) for generating orbital motion through the non-rotational structure (33) to the separation apparatus (2). The rotatable means (34) may be selected from the group consisting of a rotatable magnetic motor, a rotatable mechanical motor and the like, wherein the rotatable means (34) is firmly attached to the supporting installation (3) via an attachment (35).

Each supporting pole (31) has a pair of guiding members (32A, 32B) forming a substantially horizontal slot for securely holding the cells separation apparatus (2) in place that allows for the separation apparatus to move in an orbital motion or in a rotational motion (for example, in a centrifugal operation) as wish when the removable separation apparatus (2) is placed into the slot of the supporting installation (3). The guiding members (32A, 32B) are generally equipped with a spring-like mechanism for releasing the separation apparatus (2) when the apparatus needs to be removed from the supporting installation (3). The supporting poles (31) are so designed and configured that the separation apparatus (2) when placed into the slot of the guiding members (32A, 32B) is always at a level without undue vibration caused by the rotatable means (34).

In an illustrative example, a mechanical motor is used as the rotatable means (34). In the case of performing an orbital motion on the separation apparatus, one end of an elongate shaft (38) is secured to an axis of the mechanical motor while the other end of the elongate shaft (38) has a cam. The non-rotational drive structure (33) intimately contacts and engages the cam at an edge of the cam and is indirectly coupled to the rotatable means (34) for generating orbital motion to the second plate. Therefore, when the cam rotates, the non-rotational drive structure (33) moves in an orbital motion. The frequency of the orbital motion is related to the rotational frequency of the motor while the off-center distance of the orbital motion is related to the diameter and shape of the cam.

For operating a rotational motion on the separation apparatus, one end of an elongate shaft (38) is secured to an axis of the mechanical motor while the other end of the elongate shaft (38) is disengaged from the cam as described above. The first plate and the second plate would move together in a rotational motion as generally occurred in a centrifugal device. The upper shaft (26) is an integral part of the separation apparatus (2), wherein there are openings on the upper shaft that are in fluid communication with the separation apparatus. Each separate opening is connected to tissue fluid in-flow pump control (13), cells out-flow pump control (16), waste out-flow pump control (17), and fluid out-flow pump control (20), respectively.

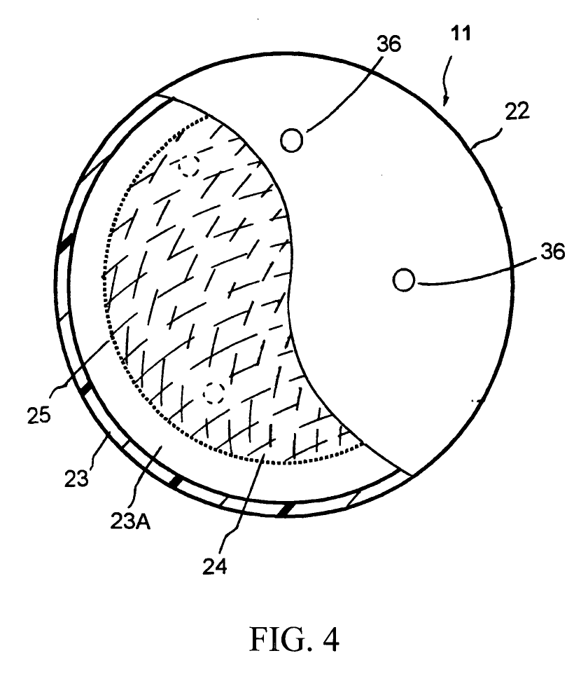

In an alternated embodiment, FIG. 4 shows a bottom view of a cells separation apparatus comprising the separation chamber (11) having a filter membrane (24). In the case of orbital motion separation, a plurality of coupling elements (36) serves as part of the non-rotational drive structure (33), wherein the coupling element (36) is detachably coupled to an exterior side of the second plate (22) of the separation chamber (11) for causing the second plate (22) to have an orbital motion in reference to a center axis of the first plate (21). The orbital motion or movement may be selected from the group consisting of clockwise movement, counterclockwise movement and a combination of the above. The off-center orbital motion or movement is generally within a range of 0.001 to 1.0 inch distance. More preferably, the off-center orbital motion is in the range of about 0.05 to 0.5 inch distance. In a further embodiment, the orbital motion may be at a frequency within a range of 500 to 50,000 cycles per minute. The frequency of the orbital motion is preferred in the range of 1,000 to 20,000 cycles per minute. The pattern of the orbital motion or movement may be selected from the group consisting of circular shape movement, oval shape movement, peanut shape movement, pear shape movement, and irregular shape movement.

For application, a cells separation method for use in separating filtrate (that is, stem and regenerative cells) from tissue fluid supply comprises the steps of (a) feeding tissue fluid supply into a separation chamber comprising filter membrane means for separating filtrate constituent from the tissue fluid supply; (b) initiating orbital motion of the filter membrane to effect separation of filtrate from tissue fluid supply; (c) collecting the filtrate constituent passing through the filter membrane; and (d) discharging a remaining constituent of the tissue fluid supply out of the separation chamber and/or adding to the out-flown cells/fluid substrate. In a preferred embodiment, the step (b) may be supplemented with or substituted by a rotational motion of the filter member to effect centrifugal separation of filtrate from tissue fluid supply. During the rotational motion operations, the coupling element (36) is detached from an exterior side of the second plate (22) of the separation chamber (11), and the first plate and second plate of the separation apparatus are moving in coordinated.

In one embodiment, the system of treating a breast of a patient comprises means for isolating the adipocytes and regenerative cells from the extracted fat tissue, wherein means for isolating may be a centrifugal separation process, a non-centrifugal separation process (preferably with an orbital motion mode), or a separation process with an alternate centrifugal separation mode and a non-centrifugal separation mode.

### EXAMPLE NO. 3 CELLS SEPARATION APPARATUS

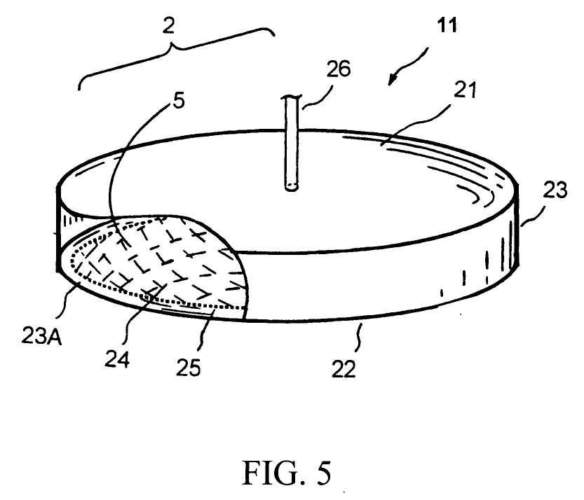

FIG. 5 shows a perspective view of the cells separation apparatus (2) comprising a separation chamber (11) having a filter membrane (24). The cells separation apparatus (2) comprises a separation chamber (11), an upper shaft (26) that has multiple fluid-flowing lumens, each lumen having a corresponding opening. The upper shaft has a first opening connected to tissue fluid in-flow pump control (13) for directing fat-plus tissue fluid supply into the chamber gap, a second opening connected to fluid out-flow pump control (20) for directing the fluid constituent passing through the filter membrane means to a collecting means, a third opening connected to waste out-flow pump control (17) for directing from the chamber gap a remaining constituent of the tissue fluid supply out of the separation chamber (11), and a fourth opening connected to cells out-flow pump control (16). In general, the openings of the upper shaft (26) are sized and configured as follows: the third opening is located at about the top portion of the separation chamber (since the fat waste is lighter than the tissue liquid); the first opening is located at about the middle portion of the separation chamber; the fourth opening is located at about next to the bottom portion of the separation chamber but above the membrane; and the second opening is located at about the bottom portion of the separation chamber but below the membrane.

The separation chamber (11) comprises a hollow interior (5) enclosed by a first plate (21), a second plate (22), and a flexible seal element (23) between the first plate (21) and the second plate (22), wherein the first plate (21) is either substantially parallel to or at an acute angle to the second plate (22) so as to form a chamber gap for the hollow interior (5). The second plate (22) comprises filter membrane means (24) for separating fluid constituent from the cells/tissue fluid supply, wherein the second plate (22) is detachably coupled to a non-rotational drive structure (33) that controls the second plate (22) in an orbital motion in reference to a center axis of the first plate (21). The chamber (11) is also detachable from the non-rotational drive structure (33).

In one preferred embodiment, the acute angle between the first plate and the second plate is in the range of 1 degree to 40 degrees so that the concentration polarization effect is minimized. The acute angle may preferably be in the range of 1 degree to 15 degrees. The acute angle may be measured from one side of the two plates to another side of the plates, from the center to the periphery of the plates or in other arbitrary manner. The flexible seal element (23, 23A) may be selected from the group consisting of silicone, polyurethane, latex, Nylon, polyvinyl chloride, polyimide, polycarbonate, polyacrylate, polymethacrylate, polystyrene, polyethylene, polypropylene, their mixture, and their copolymer. The flexible seal element of the present invention refers to a seal material that is flexible and fluid-tight so that the second plate (22) can move in an orbital motion in reference to a center axis of the first plate (21).

The filter membrane means (24) for separating the fluid constituent from the tissue fluid supply may be selected from the group consisting of nylon membrane, polycarbonate membrane, polysulfone membrane, oval pore membrane, micro-fabricated membrane, tract-edged membrane, a combination of the above and the like. In a preferred embodiment, the filter membrane means (24) is partially attached to the second plate (22) at periphery (25) of the second plate (22) so that a space below the filter membrane (24) has no fluid communication with the chamber interior (5) except through the membrane (24) itself. The periphery (25) of the second plate (22) is joined with the flexible seal element (23) by a flexible seal material (23A) so that the two plates (21, 22) can move orbitally, but not rotate, relative to each other in the orbital motion mode.

To effect an optimal filtrate filtration, the filter membrane usually has pores of a size about 0.1 to 1.0 μm. A preferred range of pore size is around 0.2 to 0.5 μm. The selection of pore size may vary with the goal of a particular separation process. The chamber gap may be between 0.001 to 0.1 inch for generating optimal local flow rate and local shear force for the separation process. A preferred range of chamber gap is about 0.02 to 0.07 inch. The optimal shear force for enhanced separation process of the present invention is a function of a combination of the chamber gap, the flow rates of the fluid supply and the outflow filtrate, and the orbital motion characteristics, wherein the orbital motion characteristics may comprise the orbiting frequency, orbiting distance, and orbiting manners. A preferred range of shear force is around 100 to 1,000 dynes/cm². Means may comprise an electromagnetic mechanism.

The cells processing step may include sampling for identifying biomarkers of adipocytes or for quality control of cell purity, cell density, and low level of inactive ingredients in the re-injectable substrate.

### EXAMPLE NO. 4 CELL CARRIER AND MATRIX

The formula consisting of breast tissue cells and cell carrier can be injected to the target site of the breast using a syringe or other fluid delivery apparatus. In one embodiment, the formula is intended to enhance revascularization in vivo. In another embodiment, the formula is intended to promote growth or multiplication of fat cells in the breast. For illustration purposes, the biocompatible matrix (that is, biomatrix) for cells to home in or adhere for intended differentiation purposes may comprise a plurality of nanoparticles or may be in nanoparticle forms that are deliverable with a syringe. Preferably, the nanoparticles are biodegradable or bioresorbable inside the breast.

Several needle types are feasible for injecting the cells-containing formula into the target breast site for tissue regeneration or breast augmentation in a one-step system. The needle is usually about 15 gauges (14 to 17 gauges) or with metal wings attached to the needle hub. The cells re-injecting step may utilize a syringe with multiple needles to simultaneously delivering stem and regenerative cells to plural sites within the breast.

The gel of the present invention may comprise methylcellulose, a temperature-sensitive polymer. Methylcellulose (MC) is a water-soluble polymer derived from cellulose, the most abundant polymer in nature. As a viscosity-enhancing polymer, it thickens solutions without precipitation over a wide pH range. A novel method using a temperature-sensitive polymer (Methylcellulose) to thermally gel aqueous alginate blended with distinct salts (CaCl₂, Na₂HPO₄, or NaCl), as a pH-sensitive hydrogel was developed for protein drug delivery (Biomacromolecules 2004; 5:1917-1925). In one preferred embodiment, stem and regenerative cells after isolation and purification steps is well-mixed to the dissolved aqueous methylcellulose or methylcellulose/alginate blended with salts at 4° C. and then gel by elevating the temperature to 37° C. when the solution is delivered in the breast. In one embodiment of the one-step system, the blend (stem cells or adipose-derived, breast tissue progenitor cells plus aqueous methylcellulose) is injected into the breast of a recipient and become a gel in vivo because of the body temperature at 37° C., a characteristic temperature for methylcellulose.

All methylcellulose compositions exhibit the classical physical behavior of cellulose ethers, changing from a solution at lower temperature to a gel at elevated temperatures. When exposing methylcellulose to an increasing temperature, the methylcellulose shows an initial period of relatively constant viscosity. Then the solution undergoes an abrupt increase in viscosity at a characteristic temperature corresponding to initiation of the first gelation phenomenon. The temperature at which gelation is initiated can be altered by varying a number of factors, including concentration of methylcellulose polymer, formulation of the aqueous solvent, additives, and heating rate. Methylcellulose was reported biocompatible with little toxicity due to degraded byproducts.

### EXAMPLE NO. 5 SELF-CONTAINED CELL ISOLATION UNIT

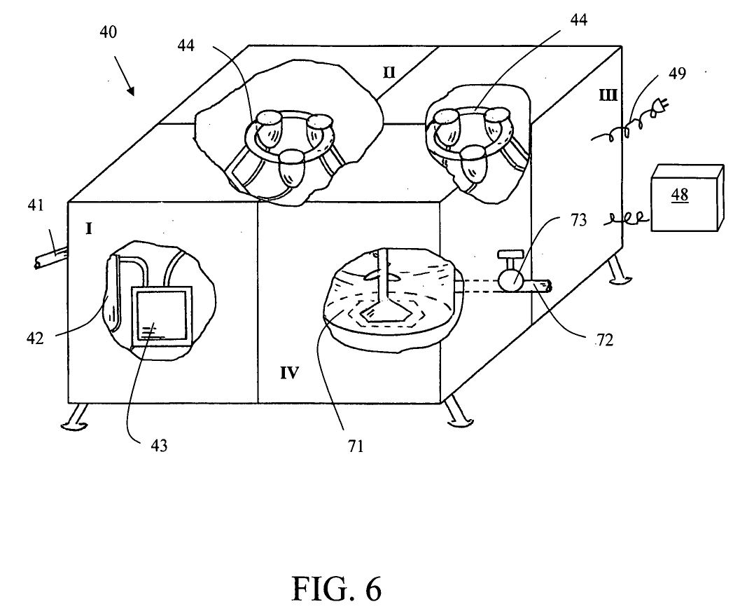

FIG. 6 shows one preferred embodiment of a cell isolation unit of the present invention for autologous cell transplantation. The unit includes an enclosure (40) having three compartments or four compartments (I to IV), an inlet port (41) for receiving fat tissue from a donor patient, an outlet port (72) for providing injectable cell compound to the patient, power sources (49), and a control mechanism (48) optionally with a CPU or other control means for operating the cell isolation unit. The unit also includes fluid containers for various cell carriers and a disposable waste container for temporarily storing any tissue waste, fat tissue or the like. In one embodiment, the enclosure may contain three compact compartments corresponding to the first three steps as shown in FIG. 1; namely, fat tissue extraction step, adipocytes isolation step, and cells processing step. The fourth step (cells re-injecting step) as shown in FIG. 1 can be performed by a physician or operator.

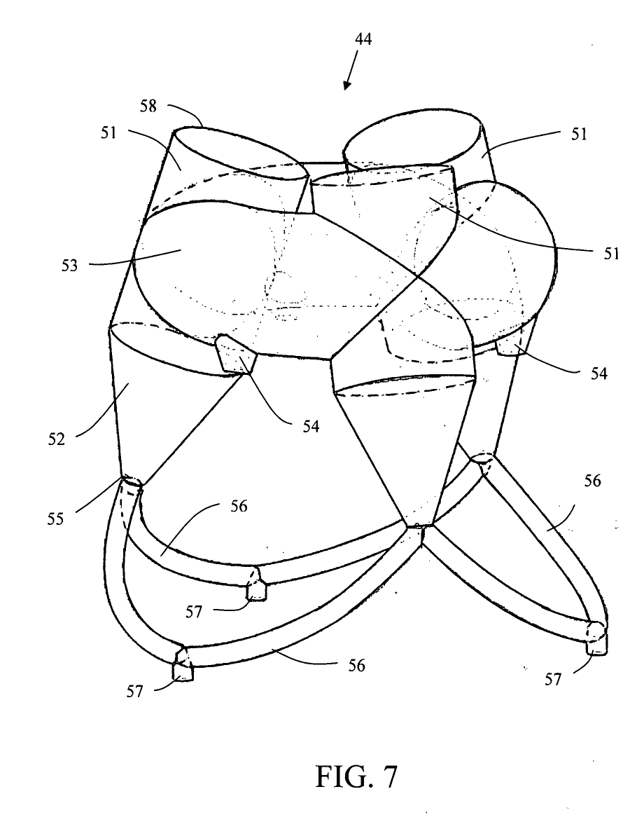

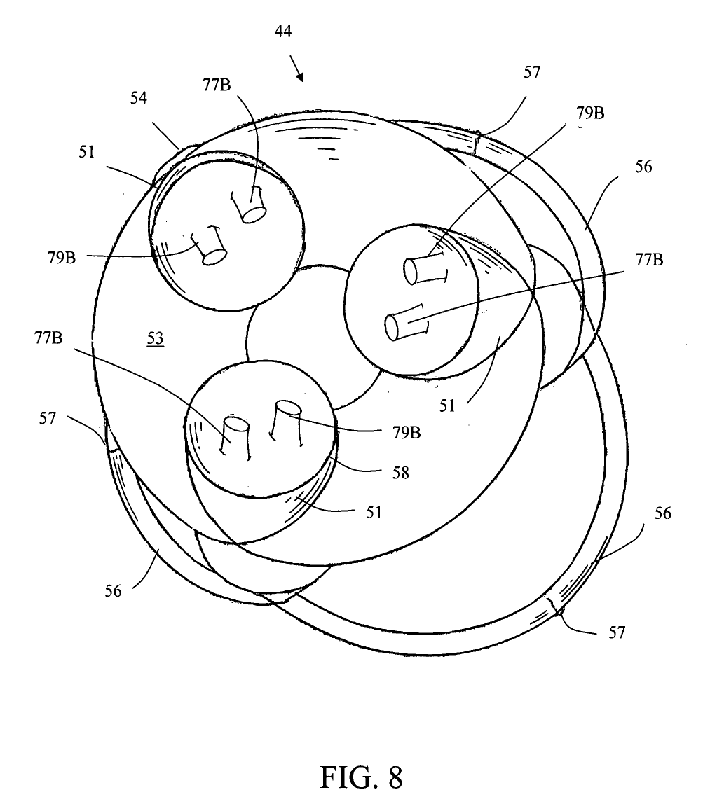

For purposes of illustration, compartment I comprises a suction assisted lipectomy instrument, comprising a tissue/fat jar (42) for the aspirate, a pump (43), and other ancillary sub-units, such as tubing, filters, vacuum gauge, bleeder valve, check valve, etc. The inlet port (41) with a valve can be connected to a liposuction cannula and is used for extracting fat from a patient, with optionally added saline or fluid. Compartment II comprises a centrifuge sub-assembly unit (44) that is detailed in FIGS. 7 and 8 for cell isolation and separation. In an exemplary embodiment of operations, a combination of fat/tissue, fluid, and saline is transported from Compartment I to Compartment II via a pump. FIG. 7 shows a front view of a centrifuge sub-assembly unit (44), whereas FIG. 8 shows a top view of the centrifuge sub-assembly unit (44).

The centrifuge sub-assembly unit comprises a plurality of cylindrical elements (51), each element having a top cover (58), a bottom cone section (52), a connecting channel (53) that is in fluid communication among elements and is located at about the middle portion of the cylindrical element, and a connecting line (56) that is in fluid communication to each and every element and is located at the bottom (55) of the cone section (52). There is further provided one male vacuum vent member (79B) with a valve and one male fluid inlet member (77B) with a valve at about the top cover (58) of each cylindrical element (51). At the intersection of any two connecting channels (53), there is provided a fat collecting pot (54) for collecting fat/tissue containing fluid. At the intersection of the connecting lines (56), there is provided a cell collecting pot (57) for collecting adipose and regenerative cells. The centrifuge sub-assembly unit (44) can be loaded on a built-in centrifuge driver that is provided in the enclosure (40) and starts the centrifuging operation under pre-determined processing conditions, optionally via the control mechanism (58). At the end of the centrifuging cycle, fat tissue and fluid are mostly collected in the fat collecting pot (54) while the cells are mostly collected in the cell collecting pot (57). Excess fat/fluid is optionally drained to the waste container for disposal later. In one embodiment, the relative volumes of the fat collecting pot (54) and the cell collecting pot (57) are pre-determined or pre-adjusted to yield a desired ratio of the collected fat/fluid to collected cells in the final cell compound.

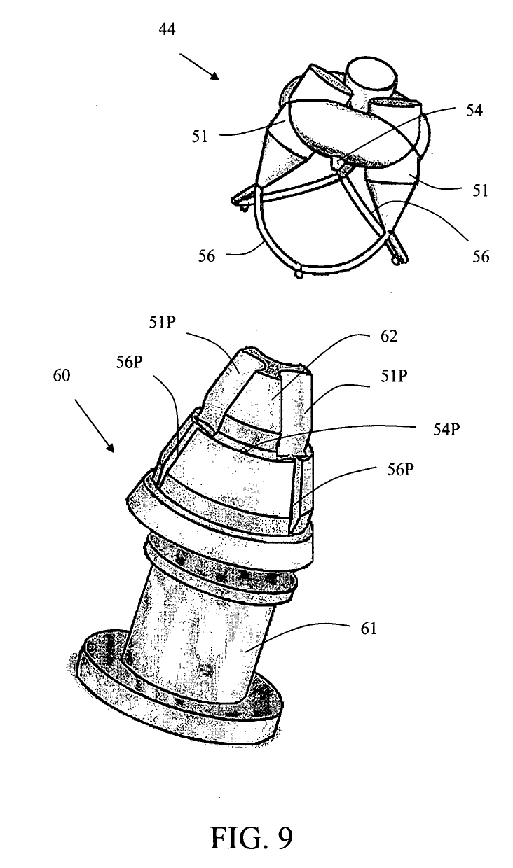

FIG. 9 shows a complete set of a centrifuge assembly unit, including the centrifuge sub-assembly unit (44) and the centrifuge driver unit (60) that securely receives the sub-assembly unit (44) during the cell isolation step. The centrifuge driver unit (60) comprises a base support (61) and a rotating member (62), wherein the rotating member has matching counterparts corresponding to those on the centrifuge sub-assembly unit (44) for securing the unit during the centrifugal operation; for example, a cylindrical element (51) and its female matching trough (51P), a fat collecting pot (54) and its female matching holder (54P), a connecting line (56) and its female matching ridge (56P). The centrifuge assembly unit of the present invention may optionally have a cap (not shown) at the top of the sub-assembly unit, wherein the cap may have a vacuum line with a valve and a fluid loading line with a valve.

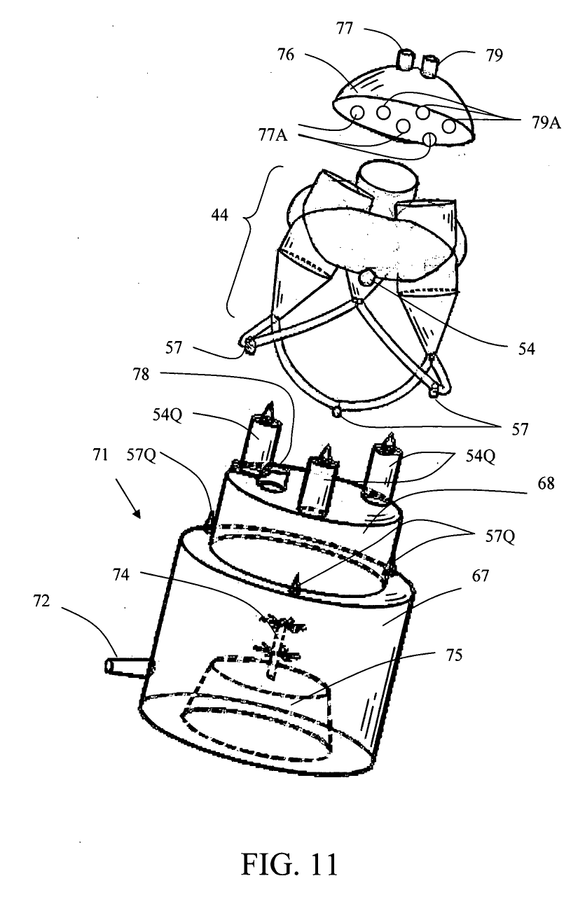

After the cell isolation step using a centrifuging process, the centrifuge sub-assembly unit (44) is thereafter transferred from Compartment II to Compartment III, where the unit (44) is loaded onto a compounder (71) or mixer, having a cap (76). There are provided a vacuum line that is connected to a vacuum or suction source (79) and a loading line (77) for taking in the extracted tissue mixture from Compartment I. The external vacuum line (79) on the cap (76) in FIG. 11 is further connected to three female vacuum receptacles (79A) at the lower portion of the cap; each vacuum receptacle (79A) matches the male vacuum vent member (79B) located at the top of the cylindrical element (51). When the cap is secured on top of the centrifuge sub-assembly unit (44), each vacuum receptacle matches its counterpart of the male vacuum vent member air-tightly. Similarly, the external fluid loading line (77) on the cap (76) in FIG. 11 is further connected to three fluid loading receptacles (77A) at the lower portion of the cap; each fluid loading receptacle (77A) matches the male fluid inlet member (77B) located at the top of the cylindrical element (51). When the cap is secured at top of the centrifuge sub-assembly unit (44), each fluid loading receptacle matches its counterpart of the male fluid inlet member air-tightly.

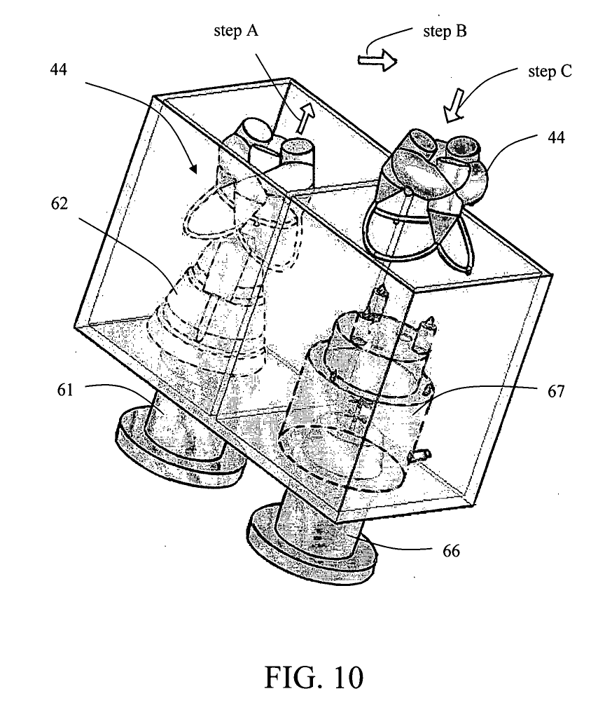

FIG. 10 shows a simulated process of transferring a centrifuge sub-assembly unit from the centrifuge set to the cell compounding set. In one embodiment, the transferring process is illustrated as indicated by step A of lifting through step B of horizontal moving to step C of lowering via a robotic arm or other mechanic arrangement. As further shown in FIG. 11, a complete set of a cell compounding assembly unit includes a compounder (71) having a stationary base construct (67) and upper adapter construct (68). The compounder (71) has matching counterparts corresponding to those on the centrifuge sub-assembly unit (44) for securing the unit during the compounding operation; for example, a fat collecting pot (54) and its female matching receiver (54Q), a cell collecting pot (57) and its female matching receiver (57Q). The compounder is maintained at a relatively constant temperature, say at about 37° C., by a wrapped-around heating pad or the like. The internal space of the compounder is kept hermetic sterile.

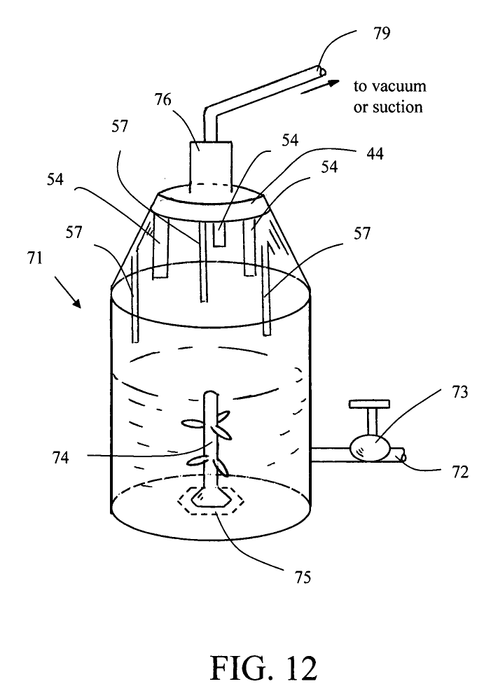

As shown in FIGS. 11 and 12, the fixed amounts of the collected fat fluid (54) and the cells (57) are delivered to the compounder that is equipped with a stirring mechanism (74). The stirring mechanism can be connected to a built-in external stirring driver (75) located within the enclosure (40). In one embodiment, the compounder can be removed for cleaning or disposed of the contents after each operation. Alternatively, the compounder (71) may be a totally disposable or a single use unit. Extra saline, sterile fluid or cell carrier can be added to the mixed cells in the compounder for later cell injection to the patient. A vacuum port (78) on the upper adapter construct (68) is provided to help emptying the contents in the centrifuge sub-assembly unit (44). In one alternate embodiment, the vacuum port (78) is connected to the vacuum or suction source (79).

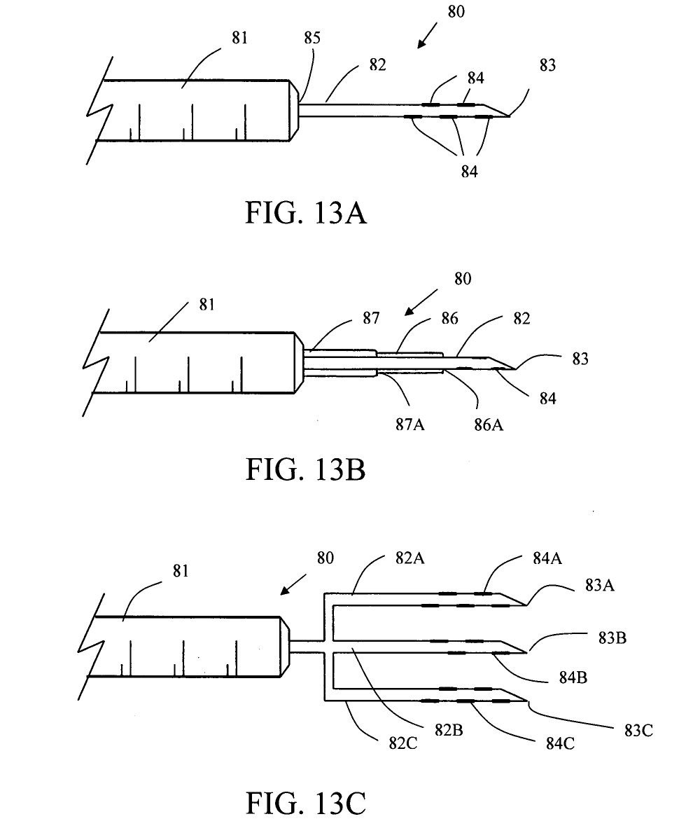

The compounded cell mixture is released and loaded to an injector via a check valve (73) and the outlet port (72). FIG. 13 shows various embodiments for the cell injector (80), each cell injector comprises a syringe-type holder (81) and a needle-type injecting element whose proximal end (85) is connected to the distal end of the holder. The injection of the compounded cell mixture can be via a pump or powered plunger, manually or automatically controlled. In one embodiment as shown in FIG. 13A, the fluid injecting element can be a single needle (82) with a sharp distal end (83) for penetration through the skin, wherein multiple fluid releasing ports (84) are in fluid communication to the holder (81) and wherein the releasing ports are spaced apart and located circumferentially around the distal section of the needle (82). In one embodiment, there is no releasing port at adjacent the sharp distal end. In one embodiment, the needle-like injecting element may have styles.

In one embodiment as shown in FIG. 13B, the fluid injecting element can have plural concentric tubes (82, 86, 87), wherein fluid from the holder (81) may release through the releasing ports (84) or through the concentric clearance (86A, 87A) between the concentric tubes. In another embodiment as shown in FIG. 13C, the fluid injecting element can have multiple spaced apart needles (82A, 82B, 82C) that are arranged parallel to each other in a 2-D or 3-D manner. The fluid releasing port (84A, 84B, 84C) on each needle (82A, 82B, 82C), respectively, is sized and configured to exert substantially about the same back pressure when it is penetrated into the breast tissue and delivers the fluid in situ.

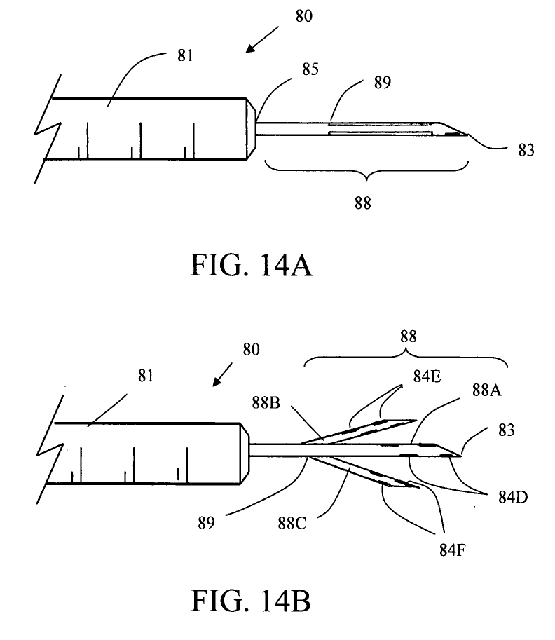

FIG. 14 shows another preferred embodiment for the cell injector (80) with splittable needles (88A, 88B, 88C). In operations, the needle sub-assembly (88) has retracted needles axially as shown in FIG. 14A similar to a single needle. After the needle sub-assembly (88) is pressed into the breast tissue and the threshold point (89) passes certain point of the breast skin, the surrounding splittable needles (88B, 88C) are splitted away from the axial needle (88A) to release cell-containing fluid compound to breast tissue at various locations (shown in FIG. 14B). As usual, the fluid releasing ports (84D, 84E, 84F) are sized, configured and spaced appropriately for optimal delivery of cell compounds.

Although the present invention has been described with reference to specific details of certain embodiments thereof, it is not intended that such details should be regarded as limitations upon the scope of the invention. Many modifications and variations are possible in light of the above disclosure.

What is claimed is:

1. A system of treating a breast of a patient, the system comprising: a) means for fat tissue extraction from a body of the patient, wherein said extracted fat tissue comprises adipocytes; b) means for isolating said adipocytes from said extracted fat tissue; c) means for compounding said adipocytes with a cell carrier; and d) means for re-injecting said compounded adipocytes to the breast of the patient autologously.

2. The system of claim 1, wherein the breast is a breast with defect.

3. The system of claim 2, wherein the defect is created mechanically.

4. The system of claim 2, wherein the defect is created chemically.

5. The system of claim 2, wherein the defect is created electromagnetically.

6. The system of claim 1, wherein the cell carrier is a gel-like biodegradable support element.

7. The system of claim 6, wherein the cell carrier and said adipocytes is compounded and configured to transiently form confluent cell-sheet like configuration.

8. The system of claim 7, wherein the confluent cell-sheet like configuration is being formed after said compound being injected into the breast.

9. The system of claim 1, wherein the cell carrier is collagenous extracellular matrix.

10. The system of claim 9, wherein the cell carrier further comprises at least one growth factors.

11. The system of claim 1, wherein the cell carrier is a temperature-sensitive deformable gel material.

12. The system of claim 1, wherein the cell carrier is methylcellulose.

13. The system of claim 1, wherein the cell carrier is a pH-sensitive deformable gel material.

14. The system of claim 1, wherein the cell carrier is chitosan/alginate complex.

15. The system of claim 1, wherein means for fat tissue extraction from the body of the patient is a liposuction process.

16. The system of claim 1, wherein means for isolating said adipocytes from said extracted fat tissue is a centrifugal separation process.

17. The system of claim 1, wherein means for isolating said adipocytes from said extracted fat tissue is a non-centrifugal separation process.

18. The system of claim 17, wherein said non-centrifugal separation process is operated with an orbital motion mode.

19. The system of claim 1, wherein means for isolating said adipocytes from said extracted fat tissue is a separation process with an alternate centrifugal separation mode and a non-centrifugal separation mode.

20. A process for treating a breast using the system of claim 1 in a real time manner.
